(19) United States
(12) Patent Application Publication
(10) Pub. No.: US 2005/0260143 A1
(43) Pub. Date: Nov. 24, 2005

(54) Toothpaste strip

(76) Inventors: Terri A. Ryan, Redford, MI (US); Sean P. Ryan, Redford, MI (US)

Correspondence Address: Arnold S. Weintraub;The Weintraub Group, P.L.C., Suite 240, 32000 Northwestern Highway, Farmington, MI, 48334, US

(21) Appl. No.: 11/132,964

(22) Filed: May 19, 2005

Related U.S. Application Data
(60) Provisional application No. 60/574,308, filed on May 19, 2004.

(51) Int. Cl. A61K 7/18; A61K 7/20; A61C 5/00

(52) U.S. Cl. 424/53; 433/217.1

(57) ABSTRACT

A system for dispensing single use quantities of a dentifrice is defined by a strip having a plurality of translucent plastic containers, each container being sealed with a peel-away strip. Each container contains a quantity of dentifrice therewithin for single dose brushing. Each container has a length and width sufficient to accommodate the insertion of a toothbrush head therewithin in order for the toothbrush head to have deposited thereon the requisite quantity of dentifrice. The strip may contain suitable indicia for indicating days of the week to ensure that toothbrushing is achieved every single day.

### CROSS REFERENC TO RELATED APPLICATIONS

The application is a completion application of co-pending U.S. Provisional Patent Application Ser. No. 60/574,308, filed May 19, 2004, for “Toothpaste Strip” the entire disclosure of which is hereby incorporated by reference.

### BACKGROUND OF THE INVENTION

1. Field of the Invention

The present invention pertains to dentifrices and dispensers therefor. More particularly, the present invention pertains to dentifrices in the form of a paste or a gel and single dose dispensers therefor.

2. Prior Art

As is know to those skilled in the art to which the present invention pertains, children, as well as adults, oftentimes, when squeezing a toothpaste or tooth gel tube leave residual amounts around the sinks or bowls creating an unsightly situation which must constantly be cleaned up. Also, present day dispensers often times have the dentifrice congeal or crust and harden around the dispensing opening or orifice.

Again, as is known to those skilled in the art to which the present invention pertains, it is often times quite difficult to entice a child to develop regular oral hygiene habits because present day dispensers do not have the allure to entice a child to want to brush his or her teeth for exactly the same problems pointed out above as well.

Furthermore, quite often, it is difficult to teach a child to apply the correct amount of paste or gel to the brush.

Thus, a major advantage would be provided by a dispensing system which enables a toothbrush to have the correct quantity of paste or gel deposited thereon and which, at the same time, teaches a child to brush his or her teeth on a regular basis.

The prior art fails in this regard such as shown in U.S. Pat. Nos. 6,524,023; 3,189,227; 6,345,733; 3,278,085; 6,260,735; 4,717,046; 2,635,788; 6,244,467 as well as Publication No. 2003/0075566.

As disclosed hereinafter, the present invention is directed to this.

### SUMMARY OF THE INVENTION

The present invention provides a dentifrice dispenser or system which, generally, comprises:
a. an elongated strip having a plurality of containers sequentially spaced therealong, each container having an access thereinto,
b. a quantity of dentifrice disposed within each container,
c. a removable seal emplaced over each container access, wherein each container is dimensioned to receive the head of a toothbrush therewithin from removing dentifrice therefrom.

The sealing means, generally, comprises a peel away foil strip.

The strip may have a plurality of spaced apart perforation disposed therealong such that each container can be separated from any other container and to devide the strip into segments.

Furthermore, each strip may contain suitable indicia to indicate a day of the week or other suitable indicia to indicate when brushing is to occur or to ensure that brushing has occurred on the appropriate day.

For a more complete understanding of the present invention reference is made to the following detailed description and accompanying drawing. In the drawing like referenced characters referred to like parts throughout the several views in which:

### BRIEF DESCRIPTION OF THE DRAWING

FIG. 1 is a front view of a dentifrice dispensing system in accordance with the present invention,

FIG. 2 is cross-sectional view taken along line 2-2 of FIG. 1, and

FIG. 3 is rear view of the dispensing system hereof.

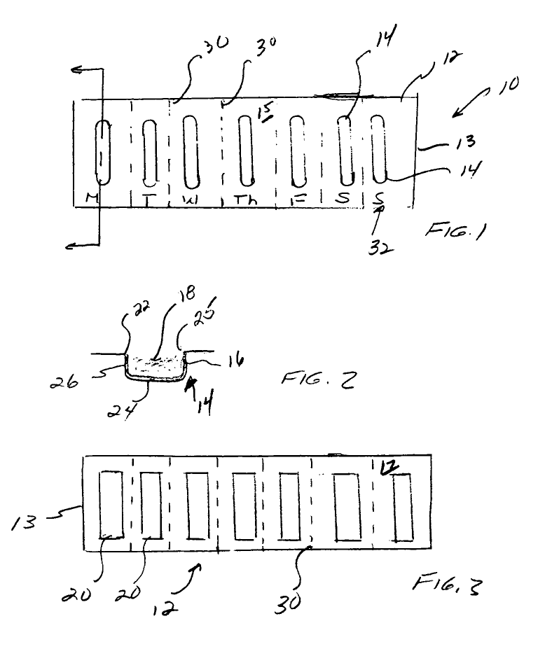

### DESCRIPTION OF THE PREFERRED EMBODIMENT

Now, and with reference to the drawing, there is depicted therein a dentifrice system or toothpaste or gel dispensing system in accordance with the present invention and, generally, denoted at 10. As shown, the system 10 hereof, generally, comprises an elongated strip 12 having a plurality of containers 14 sequentially spaced along the extent thereof. The strip 12 is a substantially flat planar member 13 having a front first side 15 and a second or opposite or back side 17. The strip 12 has a plurality of serially arranged openings 22. Each opening 22 is dimensioned to securely receive and retain an associated container 14 therewithin.

Each container 14, preferably, is a translucent member comprising an open topped receptacle 16. Each container 14 has a bottom wall 24 and an upstanding sidewall 26 integrally formed with the bottom wall 24 and an open top 25. Each container 14 is, preferably, dimensioned in length, width and depth to accommodate the size of a toothbrush head. Thus, each container 14, as well as its associated strip opening 22, is created so that the toothbrush head, be it rectangular, circular or the like, can be inserted thereinto. Each container 14 is affixed to the strip in its associated opening by any suitable means such as by sonic welding, integral formation with the strip or the like.

In accordance herewith, a quantity of dentifrice 18 such as a toothpaste, tooth gel or the like is deposited within each container. The quantity of dentifrice is predetermined to accommodate the requisite amount of dentifrice necessary for one brushing of one's teeth.

Each container 14 is dimensioned such that a toothbrush head can be inserted thereinto and have the dentifrice 18 deposited thereon. Thus, each container has a length equal to the length of the head of a toothbrush or slightly larger to enable the insertion and withdrawal of the brush head therefrom. Further, by so-dimensioning each container appropriate amount of dentifrice is deposited on to the brush head.

In order to retain the dentifrice within its respective containers, an individual sealing peel-away foil strip can be secured over the open top 25 of each container 14 to individually seal each container 14. Alternatively, a foil strip 20, or the like, may be sealingly adhered either along the length of the strip 12 to seal off the open tops 25 of the containers 14. The sealing strip is of the type well known and commercially available which can be readily peeled from and away from elongated strip.

Optimally, a plurality of sequentially spaced perforations 30 are formed along the length of the strip 12 to divide the strip into segments. This enables the containers 14 to be separated from each other and to readily be packaged for individual days, such as for travel and the like. Thus, the system comprises a plurality of individual containers 14 which can be separated from the strip 12 by tearing each individual package along respective perforations 30.

For there to be a teaching of daily hygiene, it is further contemplated that suitable indicia 32 indicating individual days of the week be disposed on the front side 15 of the strip 12 for which that package is to be used for brushing. Thus, for example, letters corresponding to the days of the week are imprinted on the front side face 15 of the strip 12. As shown, alternatively, where individual seals are used over each container, they may have a colored foil or similar suitable indicia to indicate days of the week. Likewise, each container may be a tinted translucent plastic in lieu of a transparent plastic. Preferably, the indicia is disposed between spaced perforations, i.e. indicia associated with each segment.

In order to teach both morning and night time brushing, the perforations may be placed such that two containers define a single package segment and the indicia, where used, is associated with two containers.

It is to be appreciated from the preceding that there has been described herein a toothpaste dispensing system which facilitates the teaching of a child or other person daily oral hygiene as well as providing the requisite quantity of dentifrice to effectuate proper cleaning. Furthermore, because only a single person is using the device for any one single quantity, hygienic advantages accrue since there are not multiple users involved at any given time squeezing toothpaste from the same tube.

1. A dentifrice system, comprising:
(a) an elongated strip having a plurality of containers sequentially spaced therealong,
(b) each container having an access thereto, a removable seal emplaced over each container access, a quantity of dentifrice disposed within each container, and
(c) wherein each container is dimensioned to receive the head of a toothbrush therewithin from removing dentifrice therefrom.

2. The system of claim 1 wherein the removable seal comprises a peel-away foil strip disposed over each container.

3. The system of claim 2 wherein:
each peel-away foil is of a different color to indicate a day of the week.

4. The system of claim 1 wherein:
each strip has a plurality of vertical perforations separating each container from each other, the perforations enabling separation of any one container from the rest of the strip.

5. The system of claim 4 wherein:
the perforations are associated with every other container such that there are two containers within a set for appropriate brushing day and night.

6. The system of claim 1 wherein:
each strip has a front surface and rear surface, the front surface having a plurality of indicia implaced thereon, each indicia being associated with a container to indicate an associated day of the week.

7. The system of claim 1 wherein:
each container is formed of a translucent plastic, the plastic being tinted, each tint being of a different color to identify a different day of the week.
